(19) United States
(12) Patent Application Publication
(10) Pub. No.: US 2026/0124274 A1
(43) Pub. Date: May 7, 2026

(54) METHODS OF INCREASING READTHROUGH OF A PREMATURE TERMINATION CODON

(71) Applicant: RAMOT AT TEL-AVIV UNIVERSITY LTD., Tel Aviv (IL)

(72) Inventors: Rina ROSIN-ARBESFELD, Tel Aviv (IL); Amnon WITTENSTEIN, Tel Aviv (IL); Michal CASPI, Tel Aviv (IL)

(21) Appl. No.: 19/378,469

(22) Filed: Nov. 4, 2025

Related U.S. Application Data
(60) Provisional application No. 63/715,663, filed on Nov. 4, 2024.

(51) Int. Cl. A61K 38/17; A61K 31/4245; A61K 31/436; A61K 31/496; A61K 31/506; A61K 31/519; A61K 31/7036; A61K 31/704; A61K 38/10

(52) U.S. Cl. CPC A61K 38/1709; A61K 31/4245; A61K 31/436; A61K 31/496; A61K 31/506; A61K 31/519; A61K 31/7036; A61K 31/704; A61K 38/10

(57) ABSTRACT

Methods of increasing readthrough of a premature termination codon (PTC) by a readthrough-inducing agent in a cell comprising modulating translation in the cell are provided. Methods of treating a disease characterized by a pathogenic PTC in a disease-associated gene in a subject by performing a method of the invention are also provided, as are kits comprising a readthrough-inducing agent and a translation modulating agent.

### CROSS REFERENCE TO RELATED APPLICATIONS

This application claims the benefit of priority of U.S. Provisional Patent Application No. 63/715,663, filed on Nov. 4, 2024, the contents of which are incorporated herein by reference in their entirety.

### REFERENCE TO AN ELECTRONIC SEQUENCE LISTING

The contents of the electronic sequence listing (RMT-P-035-US.xml; Size: 4,380 bytes; and Date of Creation: Nov. 3, 2025) is herein incorporated by reference in its entirety.

### FIELD OF INVENTION

The present invention is in the field of synthetic expression circuits.

### BACKGROUND OF THE INVENTION

Nonsense mutations are single nucleotide substitutions in the coding regions that result in premature termination codons (PTCs) and produce truncated, mostly non-functional proteins. A meta-analysis based on the human gene mutation databases concluded that nonsense mutations are responsible for approximately 11% of all gene aberrations associated with inheritable diseases.

Different compounds and small molecules can induce PTC readthrough, leading to misinterpretation of the PTC as a sense codon, thereby restoring protein translation. Genetic and biochemical studies have shown that these nonsense mutation readthrough agents act by binding a specific site on the rRNA, which causes the ribosome to introduce an amino acid instead of releasing the mRNA chain. Although little is known about the exact nature of the amino acid inserted or the precise readthrough mechanism, translation through the PTC often results in the expression of a full-length protein. Aminoglycoside antibiotics were the first drugs shown to induce PTC-readthrough, by enabling the misincorporation of near-cognate tRNA (nc-tRNA) at the A-site of the ribosome, leading to the expression of full-length proteins. Aminoglycosides function by a mechanism that competes with translation termination. However, the lack of specificity, modest readthrough effects, and toxicity of the aminoglycosides have led to the search for more efficient agents. Additional nonsense mutation readthrough-inducing compounds that increase protein production in several cell culture and animal disease models have been identified, but the readthrough levels were usually low, achieving no more than 5% of wild-type protein expression, and most compounds have not reached the clinic. Although nonsense mutation readthrough usually yields only a small percentage of the normal expression levels of the full-length protein, in some cases, such as in lysosomal storage disease, even 1% of normal protein function may restore a near-normal or clinically less severe phenotype, this threshold is disease and gene dependent as for cystic fibrosis (CF), it has been shown that 10-35% of CFTR activity might be needed to alleviate pulmonary morbidity significantly; in Duchenne muscular dystrophy (DMD)—1-30% of the full-length dystrophin protein is needed. It has also been demonstrated that readthrough activity can be chemically potentiated.

As a large number of genetic diseases result from nonsense mutations, identifying and developing new therapeutic strategies by better understanding the mechanism that underlines induced nonsense mutation readthrough activity is of great interest.

Adenomatous polyposis coli (APC) is a multifunctional tumor suppressor gene mutated in approximately 80% of sporadic and hereditary colorectal cancer (CRC) syndrome tumors. APC inhibits the activity of the oncogenic β-catenin protein as well as functions in cell cycle control, differentiation, and apoptosis. Mutations in APC are thought to be one of the key factors driving cancer initiation. A large number of the APC mutations are nonsense mutations, resulting in a truncated, unfunctional protein. Mechanisms that induce nonsense mutation suppression leading to restored expression of the full-length APC protein were therefore explored.

Regulating protein translation is crucial for cell survival, and thus, translational dysregulation leads to aberrant growth and tumorigenicity. A new method of suppressing translation termination at PTCs is therefore greatly needed.

### SUMMARY OF THE INVENTION

The present invention provides methods of increasing readthrough of a premature termination codon (PTC) by a readthrough-inducing agent in a cell comprising modulating translation in the cell are provided. Methods of treating a disease characterized by a pathogenic PTC in a disease-associated gene in a subject by performing a method of the invention are also provided, as are kits comprising a readthrough-inducing agent and a translation modulating agent.

According to a first aspect, there is provided a method of increasing readthrough of a premature termination codon (PTC) by a readthrough-inducing agent in a cell, the method comprising modulating translation in the cell, wherein the modulating translation comprises at least one of: inhibiting cap-dependent translation initiation in the cell, accelerating translation elongation in the cell, and inhibiting translation termination in the cell, thereby increasing readthrough of a PTC by a readthrough-inducing agent.

According to some embodiments, the readthrough-inducing agent is an antibiotic.

According to some embodiments, the antibiotic is an aminoglycoside.

According to some embodiments, the aminoglycoside is selected from geneticin, gentamicin, paromomycin and tobramycin.

According to some embodiments, the antibiotic is selected from erythromycin, azithromycin, spiramycin, josamycin, and tylosin.

According to some embodiments, the readthrough-inducing agent is selected from escin, ELX-02, NPC-14, 2,6-diaminopurine (DAP), CC-90009, amlexanox and ataluren.

According to some embodiments, the method further comprises contacting the cell with the readthrough-inducing agent.

According to some embodiments, the modulating translation comprises inhibiting cap-dependent translation initiation and comprises contacting the cell with a mammalian target of rapamycin (mTOR) inhibitor, a Eukaryotic translation initiation factor 4E (eIF4E) inhibitor, a Mitogen-activated protein kinase interacting kinase (MNK) inhibitor, or a combination thereof.

According to some embodiments, the mTOR inhibitor is selected from everolimus, temsirolimus, ridaforolimus, umirolimus, zotarolimus, AZD8055, PP242, INK128, WYE-354, OSI-027 and KU-0063794, rapamycin and Torin-1.

According to some embodiments, the eIF4E inhibitor is selected from 4E1RCat, 4E2RCat, LY2275796, SBI-756 and 4EGI-1.

According to some embodiments, the MNK inhibitor is selected from CGP57380, BAY1143269, ETC-206, MNK-11, MNK-12, MNK1/2-IN-7, SEL201, Cercosporamide, MNKI-8e and Tomivosertib.

According to some embodiments, the inhibiting cap-dependent translation initiation does not comprise administering a Ribosomal protein S6 kinase beta-1 (S6K1) inhibitor.

According to some embodiments, the modulating translation comprises accelerating translation elongation and comprises contacting the cell with a eukaryotic elongation factor 2 kinase (eEF2K) inhibitor.

According to some embodiments, the eEF2K inhibitor is selected from NH125, rottlerin, and A-484954.

According to some embodiments, inhibiting translation termination comprises contacting the cell with a eukaryotic translation termination factor 1 (eRF1) inhibitor.

According to some embodiments, the eRF1 inhibitor is selected from SRI-41315, Apidaecin, dimethyloxalylglycine (DMOG) and N-oxalylglycine (NOG).

According to another aspect, there is provided a method of treating a disease characterized by a pathogenic premature termination codon (PTC) in a disease-associated gene in a subject in need thereof, the method comprising increasing readthrough of the PTC by a readthrough-inducing agent in a cell of the subject by a method of the invention, thereby treating a disease characterized by a pathogenic PTC.

According to some embodiments, the method further comprises selecting a subject suffering from a disease characterized by a pathogenic PTC in a disease-associated gene before performing a method of the invention.

According to some embodiments, the selecting comprises receiving a disease sample from the subject and sequencing the disease-associate gene and selecting a subject whose sequence of the disease-associate gene comprises the PTC.

According to some embodiments, the disease is cancer and the disease-associated gene is a tumor suppressor gene.

According to some embodiments, the tumor suppressor gene is selected from: adenomatous polyposis coli (APC), Ataxia-telangiectasia mutated (ATM), Breast cancer type 1 susceptibility protein (BRCA1), BRCA2, cadherin-11 (CDH1), cyclin-dependent kinase inhibitor 2A (CDKN2A), Menin (MEN1), Neurofibromin (NF1), merlin (NF2), Protein patched homolog 1 (PTCH1), Phosphatase and tensin homolog (PTEN), retinoblastoma protein (RB1), SMAD family member 4, Mothers against decapentaplegic homolog 4 (SMAD4), Serine/threonine kinase 11 (STK11), p53 (TP53), tuberous sclerosis 1 (TSC1), TSC2, Von Hippel-Lindau tumor suppressor (VHL), and Wilms tumor protein (WT1).

According to some embodiments, the disease is cystic fibrosis and the disease-associated gene is cystic fibrosis transmembrane conductance regulator (CFTR), the disease is muscular dystrophy and the disease-associate gene is dystrophin (DMD), the disease is nephropathic cystinosis and the disease-associated gene is cystinosin (CTNS), the disease is epidermolysis bullosa and the disease-associated gene is laminin beta 3 (LAMB3), the disease is hereditary hypotrichosis simplex and the disease-associated gene is Lysophosphatidic acid receptor 6 (LPAR6/P2RY5) or lipase H (LIPH), or the disease is adenomatous polyposis and the disease-associated gene is APC.

According to some embodiments, the muscular dystrophy is Duchenne's muscular dystrophy (DMD).

According to some embodiments, the method comprises administering to the subject the readthrough-inducing agent.

According to another aspect, there is provided a kit comprising:
a. a readthrough-inducing agent; and
b. an agent that modulates translation.

According to some embodiments, the readthrough-inducing agent is an antibiotic.

According to some embodiments, the antibiotic is an aminoglycoside.

According to some embodiments, the aminoglycoside is selected from geneticin, gentamicin, paromomycin and tobramycin.

According to some embodiments, the antibiotic is selected from azithromycin, erythromycin, spiramycin, josamycin, and tylosin.

According to some embodiments, the readthrough-inducing agent is selected from geneticin, gentamicin, erythromycin, escin, ELX-02, NPC-14, 2,6-diaminopurine (DAP), CC-90009, amlexanox and ataluren.

According to some embodiments, the agent that modulates translation is a mammalian target of rapamycin (mTOR) inhibitor, a Eukaryotic translation initiation factor 4E (eIF4E) inhibitor, a Mitogen-activated protein kinase interacting kinase (MNK) inhibitor, or a combination thereof.

According to some embodiments, the mTOR inhibitor is selected from everolimus, temsirolimus, ridaforolimus, umirolimus, zotarolimus, AZD8055, PP242, INK128, WYE-354, OSI-027 and KU-0063794, rapamycin and Torin-1.

According to some embodiments, the eIF4E inhibitor is selected from 4E1RCat, 4E2RCat, LY2275796, SBI-756 and 4EGI-1.

According to some embodiments, the MNK inhibitor is selected from CGP57380, BAY1143269, ETC-206, MNK-11, MNK-12, MNK1/2-IN-7, SEL201, Cercosporamide, MNKI-8e and Tomivosertib.

According to some embodiments, the agent that modulates translation is a eukaryotic elongation factor 2 kinase (eEF2K) inhibitor.

According to some embodiments, the eEF2K inhibitor is selected from NH125, rottlerin, and A-484954.

According to some embodiments, the agent that modulates translation is a eukaryotic translation termination factor 1 (eRF1) inhibitor.

According to some embodiments, the eRF1 inhibitor is selected from SRI-41315, Apidaecin, dimethyloxalylglycine (DMOG) and N-oxalylglycine (NOG).

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1C: The mTOR substrate 4EBP-1 mediates the antibiotic-mediated readthrough response to serum starvation in cancerous cell lines. (1A) Colon carcinoma SW837, SW620, SW1417, and breast cancer DU4475 cell lines were supplemented with 10% or 1% serum (serum starvation) as indicated and treated with 1.5 mg/ml G418 for 24 h followed by western blot (WB) analysis using antibodies specific for APC, active β-catenin, and tubulin. The specific nonsense mutations in APC in each cell line are indicated. The graphs represent APC/tubulin band intensities (arbitrary units) calculated by Fusion-Capt analysis software. Bars represent mean values±SD of 3-5 independent experiments. One-way ANOVA tests were conducted for SW837: P<0.0001, SW620: P=0.0006, SW117: P=0.0085, and DU4475: P=0.0027. Tukey's multiple comparisons scores are shown. (1B) Volcano plot showing a two-class comparison of protein expression levels between cell lines in which serum starvation increased APC readthrough (Colo320, SW620, SW837, and DU4475-in-group) and non-responsive cell lines (SW403, SW480, SW1417 and LOVO-out-group) was conducted using the Broad Institute DepMap web portal (all the proteins available for these cells lines, in this database). (1C) Two-class comparison of protein expression of 4EBP-1 and phosphorylated forms between cell lines in which serum starvation increased APC readthrough (Colo320, SW620, SW837, and DU4475) and non-responsive cells (SW403, SW480, SW1417 and LOVO) was conducted using the Broad Institute DepMap web portal (depmap.org/portal/download/).

FIGS. 2A-2E: mTOR inhibition increases antibiotic-induced nonsense mutation readthrough. (2A) WT MEF and TSC−/− MEF cells were seeded in a 24-well plate before transfection and transiently transfected with the GFP-Stop-BFP construct (S1278X), The cells were treated with 1.5 mg/ml Gentamicin (GM) or 1 μM Rapamycin (Rap) for 24 h followed by WB analysis with the indicated antibodies. (2B) The APC 1450X reporter cell line was treated for 24 h with 500 μg/ml GM or 1 μM Rap followed by WB. The graphs show the relative GFP-BFP band intensity (normalized to GFP band intensity). Bars represent the mean values±SD from 5 independent experiments. P<0.0001. (2C-2D) (2C) Colo320 and (2D) SW403 were treated for 24 h with 500 μg/ml G418 or 1 μM Rap followed by WB. Graphs represent the intensities of the APC/tubulin or active β-catenin/tubulin bands (in arbitrary units), calculated by the Fusion-Capt analysis software. The bars represent the mean values±SD from 4-6 independent experiments. Colo320: APC P=0.0005, active β-catenin P<0.0001, SW403: APC P=0.0031, active β-catenin P=0.0048. (2E) Colo320 (one experiment) and SW403 (two independent experiments) were treated with 1.5 mg/ml G418 and 25 μM Rap for 24 h. The cells were then fixed and visualized by confocal microscopy. The graph represents the active β-catenin intensity (green) normalized to DAPI (blue) in antibiotic-treated cells. An independent script was used to quantify the RGB intensity of nuclear active β-catenin in ten independent fields of each sample. P<0.0001. Tukey's multiple comparison scores are shown. The scale bars represent 20 μM.

FIGS. 3A-3F: Reduced cap-dependent translation initiation increases antibiotic-mediated nonsense codon readthrough. (3A) The APC R1450X reporter cell line was treated for 24 h with 500 μg/ml GM and/or 50 μM 4EGI-1 (eIF4E/eIF4G Interaction Inhibitor). The graphs represent the relative GFP-BFP band intensity (normalized to GFP band intensity). Bars represent the mean values±SD from 5 independent experiments. P=0.0004. (3B-3C) (3B) Colo320 and (3C) SW403 cell lines were treated for 24 h with 500 μg/ml G418 or 50 μM 4EGI-1. The bars represent the mean values±SD from 3-6 independent experiments. Colo320: P<0.0001; SW403: P=0.0047, Tukey's multiple comparison scores are shown. (3D) The APC R1450X reporter cell line was treated for 24 h with 500 μg/ml GM and/or 30 μM Tomivosertib (MNK Inhibitor). The graphs represent the relative GFP-BFP band intensity (normalized to GFP band intensity). Bars represent the mean values±SD from 5 independent experiments. P=0.0004. (3E-3F) (3E) Colo320 and (3F) SW403 cell lines were treated for 24 h with 500 μg/ml G418 or 30 μM Tomivosertib. The bars represent the mean values±SD from 3-6 independent experiments. Colo320: P=0.0011; SW403: P=0.0014. Tukey's multiple comparison scores are shown.

FIGS. 4A-4D. Targeting different stages of the protein translation process enhances aminoglycosides-mediated nonsense codon readthrough. (4A) The APC R1450X reporter cell line was treated for 24 h with 500 μg/ml of Gentamicin (GM) and/or 100 μM A-484954 (eEF2K inhibitor, E). The graphs represent the relative GFP-BFP band intensity (normalized to GFP band intensity). Bars represent the mean values±SD from 5 independent experiments. P=0.0004. (4B) The APC R1450X reporter cell line was treated for 24 h with 500 μg/ml GM and/or 5 μM SRI-41315 (eRF1 inhibitor, T). The graphs represent the relative GFP-BFP band intensity (normalized to GFP band intensity). Bars represent the mean values±SD from 5 independent experiments. P=0.0004. (4C) Colo320 cell line was treated for 24 h with 500 μg/ml G418 and/or 100 μM A-484954. The bars represent the mean values±SD from 4-6 independent experiments. P<0.0001. (4D) Colo320 cell line was treated for 24 h with 500 μg/ml G418 and/or 5 μM SRI-41315. The bars represent the mean values±SD from 4-7 independent experiments. P<0.0001.

FIGS. 5A-5B. Targeting different stages of the protein translation process enhances readthrough mediated by non-aminoglycosides. (5A) The APC R1450X reporter cell line was treated for 24 h with 10 μM Escin or 200 μM Ataluren and 50 μM 4EGI-1, 100 μM A-484954 or 5 μM SRI-41315. (5B) The APC R1450X reporter cell line (upper blot) and Colo320 cell line (lower blot) were treated for 24 h with 300 ug/ml Erythromycin and 50 μM 4EGI-1, 100 μM A-484954 or 5 μM SRI-41315.

FIGS. 6A-6C. Induced APC nonsense mutation readthrough is specific and enhances the expression of the full-length protein. (6A) HCT116, SW48, and Colo320 cell lines were treated with 500 μg/ml G418 for 24 h followed by WB analysis using the indicated antibodies. (6B) SW403 and LOVO cell lines were treated with 1.5 mg/ml G418 for 24 h followed by WB analysis using the indicated antibodies. FS=Frameshift. (6C) Colo320 and SW480 cell lines were treated as in 6A, followed by WB analysis using antibodies specific for APC and tubulin.

FIGS. 7A-7C. Torin-1 increases antibiotic-induced nonsense mutation readthrough. (7A) The APC R1450X reporter cell line was treated for 24 h with 500 μg/ml GM and/or 500 nM Torin-1 followed by WB. The graphs show the relative GFP-BFP band intensity (normalized to GFP band intensity). Bars represent the mean values±SD from 5 independent experiments. P<0.0001. (7B-7C) (7B) Colo320 and (7C) SW403 were treated for 24 h with 500 μg/ml G418 and/or 500 nM Torin-1 followed by WB analysis. Graphs represent the intensities of the APC/tubulin or active β-catenin/tubulin bands (arbitrary units), calculated by the Fusion-Capt analysis software. The bars represent the mean values #SD from 4 independent experiments. Colo320: P<0.0001, SW403: APC P=0.0023, Active-catenin P<0.0001. Tukey's multiple comparisons scores are shown.

FIG. 8. The effects of treatment on cell survival. The APC-1450X reporter cell line, Colo320 and SW403 cell lines were treated for 24 h with 500 μg/ml GM (APC-1450X reporter cell line) or G418 (Colo320 and SW403), 500 nM Torin-1 or 1 μM Rap. PrestoBlue reagent was added to the wells and absorbance was measured after 3 h incubation at 570 and 600 nm. The bars represent the mean values±SD from 3 independent experiments for each treatment, compared to untreated cells in each cell line.

FIG. 9. APC restoration in additional CRC cell lines. SW620 and SW837 were treated for 24 h (SW620) or 48 h (SW837) with 500 μg/ml G418 and/or 500 nM Torin-1 followed by WB analysis for the indicated proteins.

FIG. 10. APC mRNA levels in the different treatments. Colo320 and SW403 cell lines were treated for 24 h with 500 μg/ml G418 and/or 1 μM Rap. Total RNA was extracted from the treated samples, converted to cDNA, and subjected to RT-qPCR analysis. APC transcript levels were analyzed. The bars represent the mean values±SD from 5 independent experiments. Two-way ANOVA (P=0.0012) with Tukey's multiple comparisons test was applied-significant scores were depicted.

FIGS. 11A-11D. Reduced cap-dependent translation initiation increases antibiotic-mediated readthrough—the effect on active β-catenin. (11A-B) (11A) Colo320 and (11B) SW403 cell lines were treated for 24 h with 500 μg/ml G418 or 50 μM 4EGI-1. The bars represent the mean values±SD from 3-6 independent experiments. Colo320: P<0.0001; SW403: active β-catenin P<0.0001. Tukey's multiple comparison scores are shown. (11C-D) (11C) Colo320 and (11D) SW403 cell lines were treated for 24 h with 500 μg/ml G418 or 30 μM Tomivosertib. The bars represent the mean values±SD from 3-6 independent experiments. Colo320: P=0.0073; SW403: P=0.0058. Tukey's multiple comparison scores are shown.

FIG. 12. S6K1 phosphorylation is not involved in antibiotic-mediated nonsense mutation readthrough. The APC 1450X reporter cell line was treated for 24 h with 500 μg/ml GM and 0.5, 1, 5, 10, or 20 μM PF-4708671 (S6K1 inhibitor) followed by WB analysis using the indicated antibodies. The graphs represent the relative GFP-BFP band intensity (normalized to GFP band intensity; with or without 20 μM PF-4708671), calculated by the Fusion-Capt analysis software. The bars represent the mean values±SD from 4 independent experiments.

FIG. 13. Additional termination inhibitors enhance antibiotic-mediated nonsense readthrough. The APC 1450X reporter cell line was treated for 24 h with 500 μg/ml GM and 100 μg/ml Apidaecin or 500 μM N-Oxalylglycine (NOG). The bars represent the relative GFP-BFP band intensity (normalized to GFP band intensity) mean values±SD from 3-4 independent experiments. P<0.0001.

### DETAILED DESCRIPTION OF THE INVENTION

The present invention, in some embodiments, provides methods of increasing readthrough of a premature termination codon (PTC) by a readthrough-inducing agent in a cell comprising modulating translation in the cell are provided. Methods of treating a disease characterized by a pathogenic PTC in a disease-associated gene in a subject by performing a method of the invention are also provided, as are kits comprising a readthrough-inducing agent and a translation modulating agent.

In this study, the inventors show that inhibiting translation initiation by disturbing the translation initiation complex, as well as targeting the elongation or termination stages of protein translation, increases induced nonsense suppression. The results expose the complex relationship between nonsense mutation readthrough and the translation machinery and offer new approaches to improve full-length protein production from genes containing nonsense mutations.

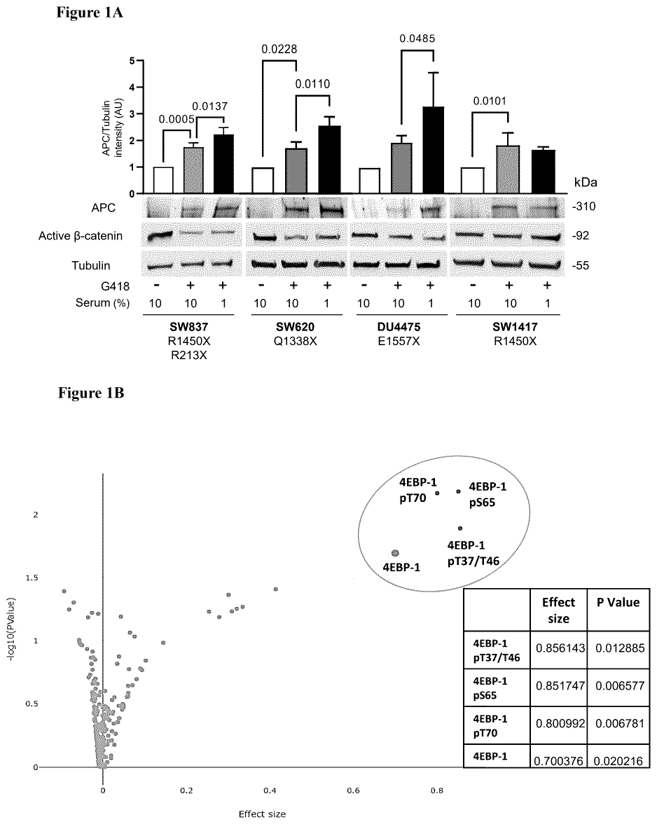

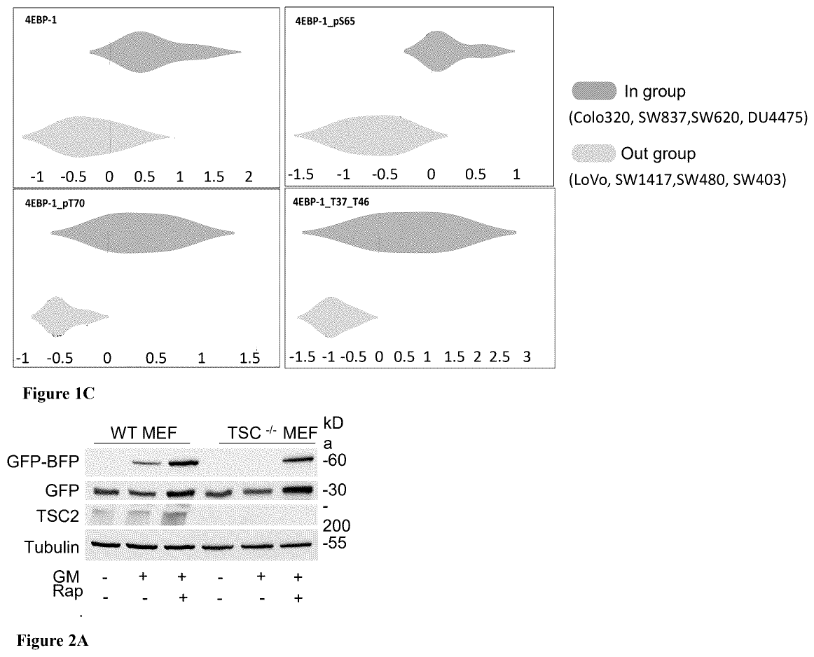

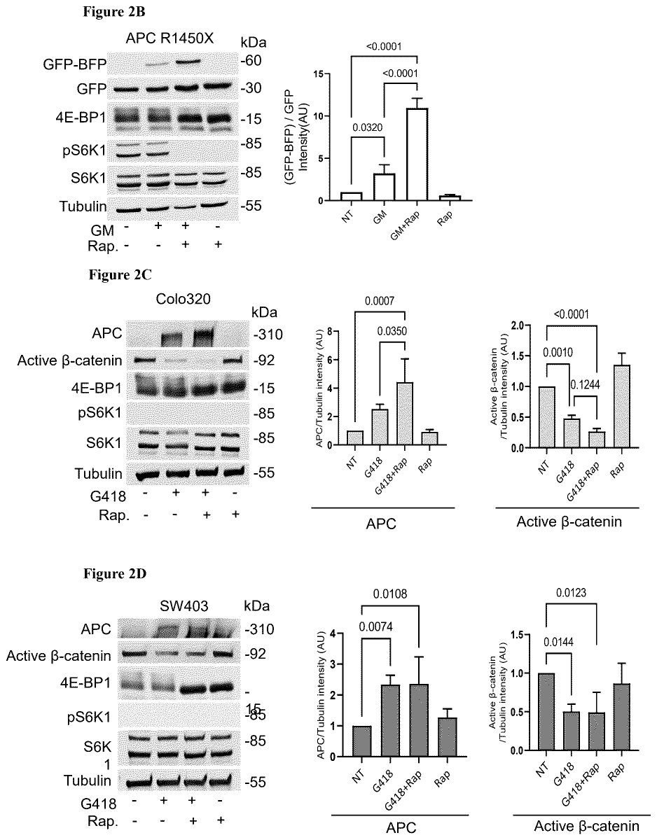

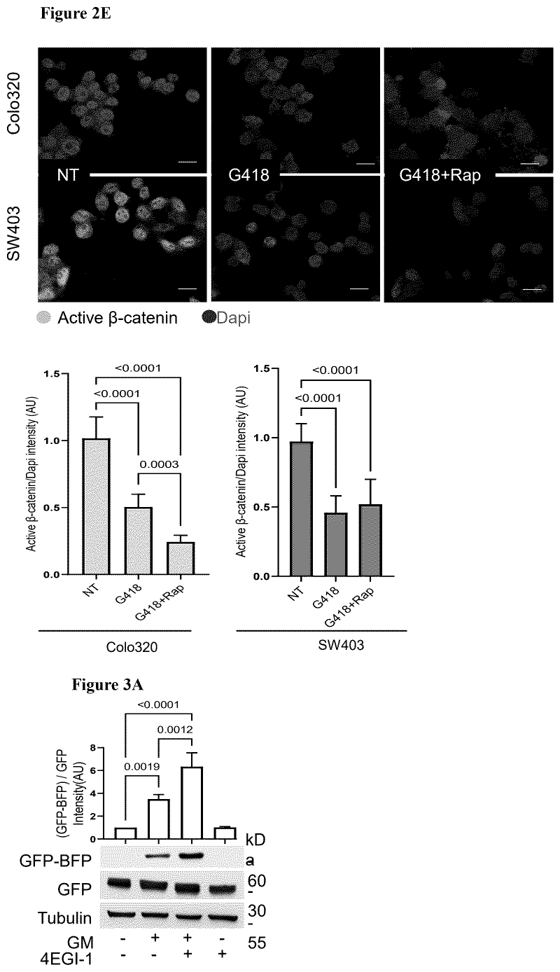

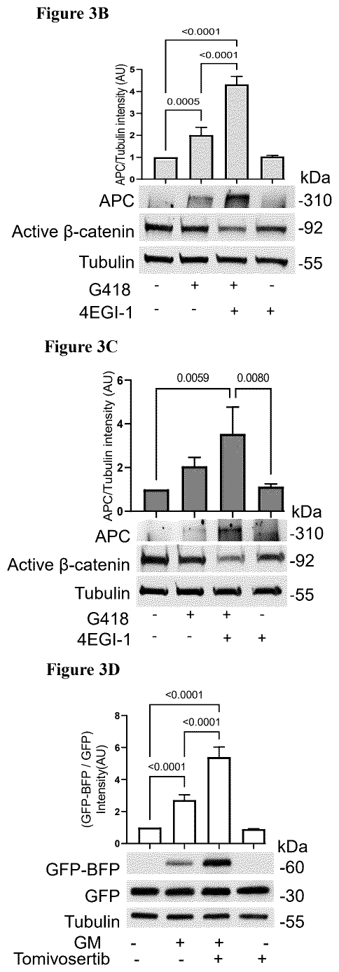

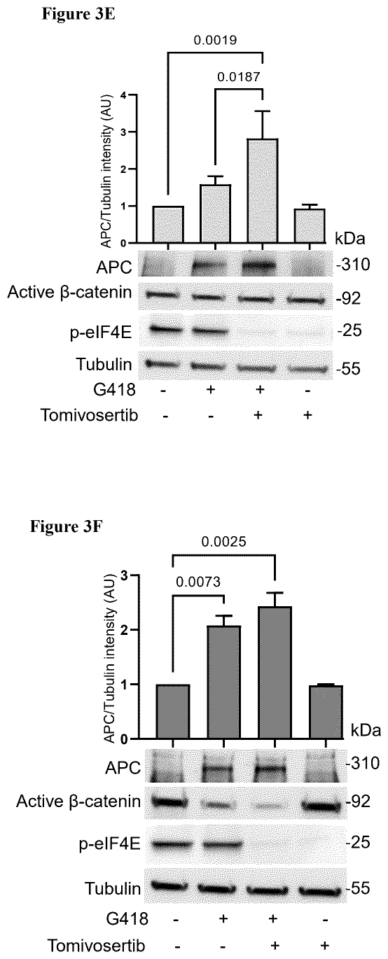

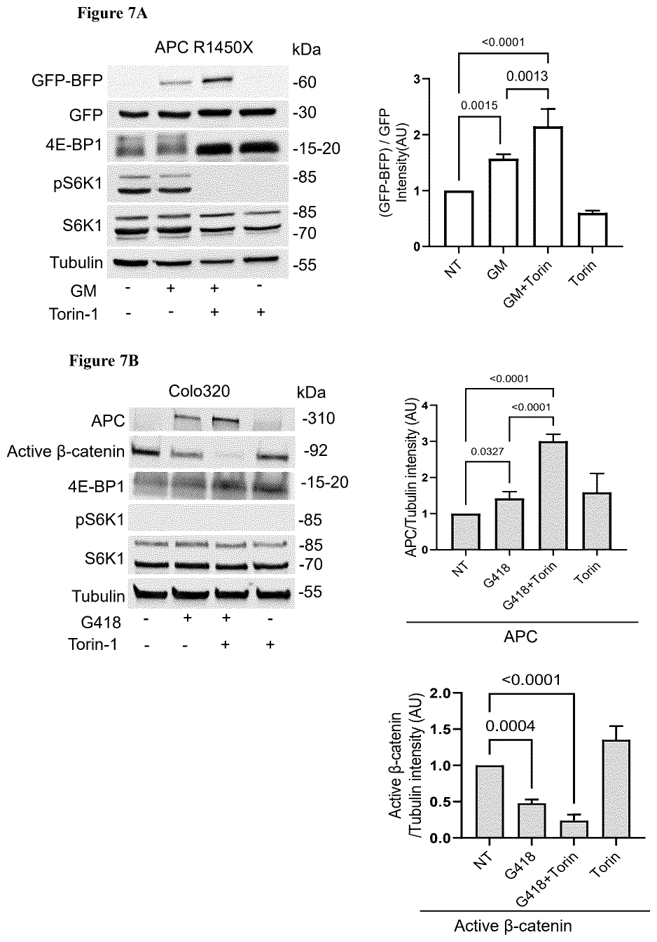

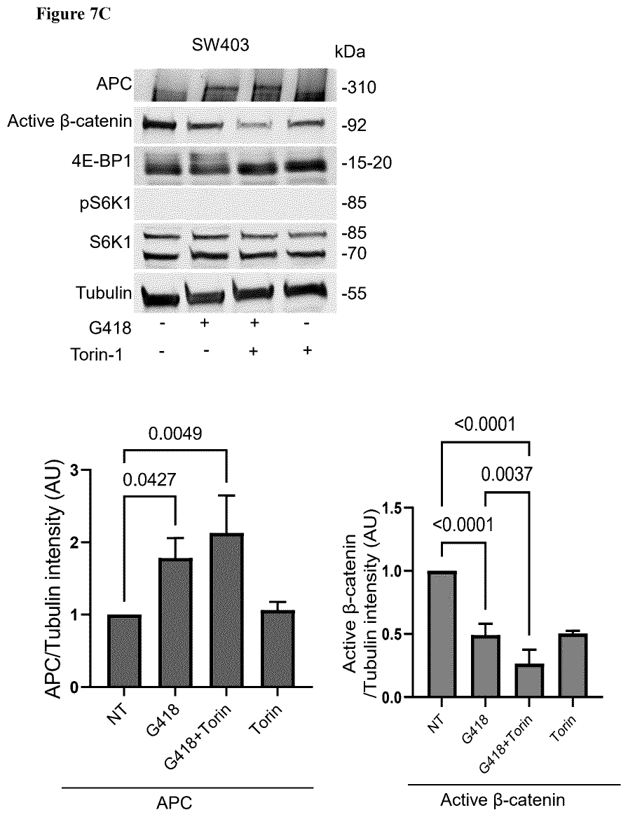

The invention is based, at least in part, on the surprising implication of mTOR substrate 4EBP-1 in antibiotic-mediated nonsense suppression. 4EBP-1, along with several of its phosphorylated forms, were shown to be differentially expressed in cells that either responded or did not respond to serum deprivation-induced nonsense mutation readthrough (FIG. 1). The eIF4E/eIF4G translation initiation complex is active when mTOR-dependent phosphorylation of 4EBP-1 occurs. The small molecule 4EGI-1, which inhibits cap-dependent translation initiation by binding to eIF4E and disrupting the eIF4E/eIF4G association, efficiently increases antibiotic-mediated readthrough. 4EGI-1 is of particular interest since it has anti-tumorigenic activity and reduces the growth of human cancer xenografts in vivo [82]. The finding that antibiotic-mediated nonsense suppression is susceptible to translation initiation rate was further demonstrated by MNK inhibition that impedes eIF4E phosphorylation and inhibits translation initiation (FIG. 3D-3F). Numerous MNK inhibitors (e.g., BAY1143269, Tomivosertib, CGP 57380, and ETC-206) have been tested in various clinical trials as a potential therapeutic strategy for cancer treatment. The results provided herein also demonstrate that mTOR inhibitors such as Torin-1 and Rapamycin, which lead to decreased translation initiation (by inhibiting the phosphorylating of 4EBP-1) augmented PTC readthrough as examined in a reporter fusion protein and the endogenous APC protein (FIGS. 2 and 7). Aspects of this invention are disclosed by the Inventors in Wittenstein et. al., “Nonsense mutation suppression is enhanced by targeting different stages of the protein translation process”, PLOS Biol, 2023 Nov. 9; 21 (11): e3002355, the contents of which is hereby incorporated by reference in its entirety.

By a first aspect, there is provided a method of increasing readthrough of a premature termination codon (PTC) by a readthrough-inducing agent in a cell, the method comprising modulating translation in the cell, thereby increasing readthrough of a PTC by a readthrough-inducing agent.

In some embodiments, the method is an in vivo method. In some embodiments, the method is a therapeutic method. In some embodiments, the cell is in a subject. In some embodiments, the cell is a disease cell. In some embodiments, the cell is a diseased cell. In some embodiments, the cell is a cancerous cell. In some embodiments, the cell is a muscle cell. In some embodiments, the method is an ex vivo method. In some embodiments, the method is an in vitro method. In some embodiments, the cell is in culture. In some embodiments, the cell is for use in adoptive cell transfer. In some embodiments, the cell is from the subject.

By another aspect, there is provided an agent that modulates translation for use in increasing readthrough of a premature termination codon (PTC) by a readthrough-inducing agent in a subject.

By another aspect, there is provided an agent that modulates translation for use in combination with a readthrough-inducing agent for increasing readthrough of a premature termination codon (PTC) in a subject.

In some embodiments, modulating translation comprises inhibiting cap-dependent translation. In some embodiments, the inhibiting is in the cell. In some embodiments, the inhibiting is in the subject. In some embodiments, inhibiting cap-dependent translation is inhibiting cap-dependent translation initiation. In some embodiments, inhibiting cap-dependent translation comprises contacting the cell with a mammalian target of rapamycin (mTOR) inhibitor. In some embodiments, inhibiting cap-dependent translation comprises administering to the subject an mTOR inhibitor. In some embodiments, the mTOR inhibitor is selected from everolimus, temsirolimus, ridaforolimus, umirolimus, zotarolimus, AZD8055, PP242, INK128, WYE-354, OSI-027 and KU-0063794, rapamycin and Torin-1. In some embodiments, the mTOR inhibitor is selected from rapamycin and Torin-1. In some embodiments, the mTOR inhibitor is rapamycin. In some embodiments, the mTOR inhibitor is Torin-1.

Rapamycin is also known as sirolimus and is a macrolide compound that is a specific inhibitor of mTOR. Rapamycin is known to modulate translation processes and enhance the readthrough of premature termination codons (PTCs). Torin-1 is a synthetic inhibitor of mTOR. It targets both mTORC1 and mTORC2 complexes, blocking ATP-binding to mTOR and thereby inhibiting cell growth and proliferation. Both rapamycin and Torin-1 are commercially available from a variety of sellers, as are the other recited mTOR inhibitors.

In some embodiments, inhibiting cap-dependent translation comprises contacting the cell with a Eukaryotic translation initiation factor 4E (eIF4E) inhibitor. In some embodiments, inhibiting cap-dependent translation comprises administering to the subject an eIF4E inhibitor. In some embodiments, the eIF4E inhibitor is selected from 4E1RCat, 4E2RCat, LY2275796, SBI-756 and 4EGI-1. In some embodiments, the eIF4E inhibitor is selected from 4E1RCat, 4E2RCat, LY2275796, SBI-756 and 4EGI-1. In some embodiments, the eIF4E inhibitor is 4EGI-1. 4EGI-1 is a small-molecule inhibitor that targets the eIF4E) It disrupts the interaction between eIF4E and eIF4G, inhibiting cap-dependent translation initiation. 4EGI-1 is commercially available from a variety of sellers, as are the other recited eIF4E inhibitors.

In some embodiments, inhibiting cap-dependent translation comprises contacting the cell with a Mitogen-activated protein kinase interacting kinase (MNK) inhibitor. In some embodiments, inhibiting cap-dependent translation comprises administering to the subject an MNK inhibitor. In some embodiments, the MNK inhibitor is selected from CGP57380, BAY1143269, ETC-206, MNK-11, MNK-12, MNK1/2-IN-7, SEL201, Cercosporamide, MNKI-8e and Tomivosertib. In some embodiments, the MNK inhibitor is selected from MNKI-8e and Tomivosertib. In some embodiments, the MNK inhibitor is Tomivosertib. Tomivosertib is a potent and selective inhibitor of the mitogen-activated protein kinase interacting kinases 1 and 2 (MNK1/2). It works by impeding the phosphorylation of eukaryotic translation initiation factor 4E (eIF4E), thereby inhibiting cap-dependent translation initiation. Tomivosertib is commercially available from a variety of sellers, as are the other recited MNK inhibitors.

In some embodiments, inhibiting cap-dependent translation does not comprise inhibiting Ribosomal protein S6 kinase beta-1 (S6K1). In some embodiments, inhibiting cap-dependent translation does not comprise contacting the cell with an S6KI inhibitor. In some embodiments, inhibiting cap-dependent translation does not comprise administering to the subject an S6KI inhibitor. In some embodiments, the agent that modulates translation is not an S6KI inhibitor. In some embodiments, an agent that modulates translation is a modulator of translation. In some embodiments, the modulator of translation is not an S6KI inhibitor. In some embodiments, an agent that modulates translation is an inhibitor. In some embodiments, the inhibitor of the invention is not an S6KI inhibitor. In some embodiments, the inhibitor is an mTOR inhibitor. In some embodiments, the inhibitor is an eIF4E inhibitor. In some embodiments, the inhibitor is an MNK inhibitor. In some embodiments, the inhibitor is an eEF2K inhibitor. In some embodiments, the inhibitor is an eRF1 inhibitor.

In some embodiments, modulating translation comprises accelerating translation elongation. In some embodiments, the accelerating is in the cell. In some embodiments, the accelerating is in the subject. In some embodiments, accelerating translation elongation comprises contacting the cell with a eukaryotic elongation factor 2 kinase (eEF2K) inhibitor. In some embodiments, accelerating translation elongation comprises administering to the subject an eEF2K inhibitor. In some embodiments, the eEF2K inhibitor is selected from NH125, rottlerin, and A-484954. In some embodiments, the eEF2K inhibitor is A-484954. A-484954 is a highly selective inhibitor of eukaryotic elongation factor 2 kinase (eEF2K). By inhibiting eEF2K, A-484954 enhances the activity of eukaryotic elongation factor 2 (eEF2), leading to accelerated translation elongation. A-484954 is commercially available from a variety of sellers, as are the other recited eEF2K inhibitors.

In some embodiments, modulating translation comprises reducing translation termination. In some embodiments, the reducing is in the cell. In some embodiments, the reducing is in the subject. In some embodiments, reducing translation termination comprises inhibiting, degrading or depleting eukaryotic translation termination factor 1 (eRF1). In some embodiments, reducing translation termination comprises inhibiting eRF1. In some embodiments, reducing translation termination comprises depleting eRF1. In some embodiments, reducing translation termination comprises degrading eRF1. In some embodiments, the depleting is in the cell. In some embodiments, the depleting is in the subject. In some embodiments, reducing translation termination comprises contacting the cell with an eRF1 inhibitor. In some embodiments, reducing translation termination comprises administering to the subject an eRF1 inhibitor. In some embodiments, the eRF1 inhibitor is selected from SRI-41315, Apidaecin, dimethyloxalylglycine (DMOG) and N-oxalylglycine (NOG). In some embodiments, the eRF1 inhibitor is selected from SRI-41315, Apidaecin and NOG. In some embodiments, the eRF1 inhibitor is selected from Apidaecin and NOG. In some embodiments, the eRF1 inhibitor is SRI-41315. In some embodiments, the eRF1 inhibitor is Apidaecin. In some embodiments, the eRF1 inhibitor is NOG. SRI-41315 is a small molecule that depletes eRF1 levels through a proteasome-mediated degradation pathway. Apidaecin is an antimicrobial peptide derived from honeybees. It inhibits bacterial growth by interfering with protein synthesis and specifically inhibiting eRF1. N-oxalylglycine (NOG) is a compound known to inhibit the hydroxylation of certain proteins including eRF1. SRI-41315, apidaecin, DMOG and NOG are all commercially available from a variety of sellers.

In some embodiments, the method further comprises contacting the cell with the readthrough-inducing agent. In some embodiments, the method further comprises administering to the subject the readthrough-inducing agent. In some embodiments, the readthrough-inducing agent is an antibiotic. In some embodiments, the antibiotic is an aminoglycoside. In some embodiments, the aminoglycoside is selected from geneticin, gentamicin, paromomycin and tobramycin. In some embodiments, the aminoglycoside is selected from geneticin, and gentamicin. In some embodiments, the aminoglycoside is geneticin. In some embodiments, the aminoglycoside is gentamicin. In some embodiments, the antibiotic is selected from erythromycin, azithromycin, spiramycin, josamycin, and tylosin. In some embodiments, the antibiotic is selected from erythromycin and azithromycin. In some embodiments, the antibiotic is erythromycin. In some embodiments, the antibiotic is azithromycin. In some embodiments, the antibiotic is selected from geneticin, gentamicin, paromomycin, tobramycin, erythromycin, azithromycin, spiramycin, josamycin, and tylosin. In some embodiments, the antibiotic is selected from geneticin, gentamicin, erythromycin, and azithromycin. In some embodiments, the antibiotic is selected from gentamicin and erythromycin.

In some embodiments, the readthrough-inducing agent is not an antibiotic. In some embodiments, the readthrough-inducing agent is selected from escin, ELX-02, NPC-14, 2,6-diaminopurine (DAP), CC-90009, amlexanox and ataluren. In some embodiments, the readthrough-inducing agent is selected from escin and ataluren. In some embodiments, the readthrough-inducing agent is escin. In some embodiments, the readthrough-inducing agent is ataluren. In some embodiments, the readthrough-inducing agent is selected from gentamicin, erythromycin, escin and ataluren. In some embodiments, the readthrough-inducing agent is selected from geneticin, gentamicin, erythromycin, escin and ataluren. In some embodiments, the readthrough-inducing agent is selected from geneticin, gentamicin, erythromycin, azithromycin, escin and ataluren.

By another aspect, there is provided a method of treating a disease in a subject in need thereof, the method comprising increasing readthrough of a premature termination codon (PTC) by a readthrough-inducing agent in the subject by a method of the invention, thereby treating a disease.

By another aspect, there is provided an agent that modulates translation for use in treating a disease in a subject.

By another aspect, there is provided an agent that modulates translation for use in combination with a readthrough-inducing agent for treating a disease in a subject.

In some embodiments, the disease is characterized by a PTC. In some embodiments, the PCT is a pathogenic PTC. In some embodiments, the PTC causes the disease. In some embodiments, the PTC contributes to the pathology of the disease. In some embodiments, the PTC is in a disease-associated gene. In some embodiments, the disease-associated gene is a disease-causing gene.

In some embodiments, the disease is cancer. In some embodiments, the disease is cancer and the gene comprising a PTC is a tumor suppressor gene. In some embodiments, the disease associated gene is a tumor suppressor gene. In some embodiments, the cancer is a solid cancer. In some embodiments, the cancer is a hematopoietic cancer. In some embodiments, the cancer is selected from hepato-biliary cancer, cervical cancer, urogenital cancer, anogenital cancer, prostate cancer, thyroid cancer, ovarian cancer, nervous system cancer, ocular cancer, lung cancer, soft tissue cancer, bone cancer, pancreatic cancer, bladder cancer, skin cancer, intestinal cancer, hepatic cancer, rectal cancer, colorectal cancer, esophageal cancer, gastric cancer, gastroesophageal cancer, breast cancer, renal cancer, skin cancer, head and neck cancer, leukemia and lymphoma. In some embodiments, the cancer is colorectal cancer. In some embodiments, the cancer is breast cancer. In some embodiments, the cancer is a carcinoma. In some embodiments, the carcinoma is an adenocarcinoma. In some embodiments, the cancer is an epithelial cancer.

In some embodiments, the tumor suppressor gene is selected from adenomatous polyposis coli (APC), Ataxia-telangiectasia mutated (ATM), Breast cancer type 1 susceptibility protein (BRCA1), BRCA2, cadherin-11 (CDH1), cyclin-dependent kinase inhibitor 2A (CDKN2A), Menin (MEN1), Neurofibromin (NF1), merlin (NF2), Protein patched homolog 1 (PTCH1), Phosphatase and tensin homolog (PTEN), retinoblastoma protein (RB1), SMAD family member 4, Mothers against decapentaplegic homolog 4 (SMAD4), Serine/threonine kinase 11 (STK11), p53 (TP53), tuberous sclerosis 1 (TSC1), TSC2, Von Hippel-Lindau tumor suppressor (VHL), and Wilms tumor protein (WT1). In some embodiments, the tumor suppressor gene is selected from APC, TSC1 and TSC2. In some embodiments, the tumor suppressor gene is APC. In some embodiments, the tumor suppressor gene is selected from TSC1 and TSC2. In some embodiments, the tumor suppressor gene is TSC1. In some embodiments, the tumor suppressor gene is TSC2. In some embodiments, the cancer is colorectal cancer and the tumor suppressor gene is APC. In some embodiments, the cancer is colorectal cancer and the tumor suppressor gene is selected from TSC1 and TSC2. In some embodiments, the cancer is breast cancer and the tumor suppressor gene is APC. In some embodiments, the cancer is breast cancer and the tumor suppressor gene is selected from TSC1 and TSC2.

In some embodiments, the disease is a genetic disease. In some embodiments, the disease is cystic fibrosis (CF). In some embodiments, the disease is CF and the disease-associated gene is cystic fibrosis transmembrane conductance regulator (CFTR). In some embodiments, the disease is muscular dystrophy. In some embodiments, the muscular dystrophy is Duchenne's muscular dystrophy. In some embodiments, the muscular dystrophy is Becker muscular dystrophy. In some embodiments, the disease is muscular dystrophy and the gene is dystrophin (DMD). In some embodiments, the disease is nephropathic cystinosis. In some embodiments, the disease is nephropathic cystinosis and the disease-associated gene is cystinosin (CTNS). In some embodiments, the disease is epidermolysis bullosa. In some embodiments, the disease is epidermolysis bullosa and the disease-associated gene is laminin beta 3 (LAMB3). In some embodiments, the disease is hereditary hypotrichosis simplex. In some embodiments, the disease is hereditary hypotrichosis simplex and the disease-associated gene is selected from Lysophosphatidic acid receptor 6 (LPAR6/P2RY5) and lipase H (LIPH). In some embodiments, the disease is adenomatous polyposis. In some embodiments, the disease is adenomatous polyposis and the disease-associated gene is APC.

In some embodiments, the method further comprises administering the readthrough-inducing agent to the subject. In some embodiments, the method further comprises selecting a subject suffering from the disease. In some embodiments, the method further comprises selecting a subject suffering from a disease characterized by a PTC. In some embodiments, the selecting is selecting a subject suffering from a disease characterized by a PTC of a disease-associated gene. In some embodiments, the selecting is before performing the method. In some embodiments, the method is a method of selecting and treating. In some embodiments, the selecting comprises determining the presence of the PTC. In some embodiments, the selecting comprises confirming the presence of the PTC.

In some embodiments, the selecting comprises receiving a sample from the subject. In some embodiments, the selecting comprises obtaining a sample from the subject. In some embodiments, the sample is a disease sample. In some embodiments, the sample comprises cells. In some embodiments, the sample comprises DNA. In some embodiments, the sample is a bodily fluid. In some embodiments, the sample is a tissue sample. In some embodiments, the sample is a biopsy. In some embodiments, the biopsy is a liquid biopsy. In some embodiments, the bodily fluid is selected from at least one of: blood, serum, plasma, gastric fluid, intestinal fluid, saliva, bile, tumor fluid, breast milk, urine, interstitial fluid, cerebral spinal fluid and stool. In some embodiments, the selecting further comprises isolating DNA from the sample. In some embodiments, the selecting further comprises extracting DNA from the sample. In some embodiments, the selecting further comprises sequencing the sample. In some embodiments, the selecting further comprises sequencing the DNA from the sample. In some embodiments, the sequencing is sequencing the disease-associated gene. In some embodiments, the sequencing is sequencing a gene that may contain a PTC. In some embodiments, the method comprises selecting a subject with a disease-associated gene comprising the PTC. In some embodiments, the method comprises selecting a subject whose sequence of the disease-associated gene comprises the PTC. In some embodiments, the method comprises selecting a subject whose sequenced DNA from the sample comprises the PTC. In some embodiments, the method comprises selecting a subject whose sequenced DNA from the sample comprises the disease-associated gene comprising the PTC. It is thus clear that the instant method pertains to treating a patient population for which the combination of the readthrough agent and the translational modifier are particularly suited. Some of the translational modifiers may already be used for treating the disease (i.e., they may be known for treating cancer), but their specific use for treating cancer patients with a disease PTC is not known and certainly not for treating in combination with a readthrough agent.

As used herein, the terms “administering,” “administration,” and like terms refer to any method which, in sound medical practice, delivers a composition containing an active agent to a subject in such a manner as to provide a therapeutic effect. One aspect of the present subject matter provides for oral administration of a therapeutically effective amount of a composition of the present subject matter to a patient in need thereof. Other suitable routes of administration can include parenteral, subcutaneous, intravenous, intramuscular, or intraperitoneal.

The dosage administered will be dependent upon the age, health, and weight of the recipient, kind of concurrent treatment, if any, frequency of treatment, and the nature of the effect desired.

By another aspect, there is provided a kit comprising a readthrough-inducing agent and an agent that modulates translation.

In some embodiments, the kit is for use in a method of the invention. In some embodiments, the kit is for use in increasing readthrough of a premature termination codon (PTC). In some embodiments, the kit is for use in treating a disease characterized by a PTC.

In some embodiments, an agent that modulates translation is an inhibitor. In some embodiments, an agent that modulates translation inhibits cap-dependent translation. In some embodiments, an agent that modulates translation does not inhibit S6K1. In some embodiments, an agent that modulates translation is not an S6K1 inhibitor. In some embodiments, an agent that inhibits cap-dependent translation does not inhibit S6K1. In some embodiments, an agent that inhibits cap-dependent translation is not an S6K1 inhibitor.

In some embodiments, an agent that inhibits cap-dependent translation is an agent that inhibits mTOR. In some embodiments, an agent that inhibits mTOR is an mTOR inhibitor. In some embodiments, the mTOR inhibitor is selected from everolimus, temsirolimus, ridaforolimus, umirolimus, zotarolimus, AZD8055, PP242, INK128, WYE-354, OSI-027 and KU-0063794, rapamycin and Torin-1. In some embodiments, the mTOR inhibitor is selected from rapamycin and Torin-1. In some embodiments, the mTOR inhibitor is rapamycin. In some embodiments, the mTOR inhibitor is Torin-1.

In some embodiments, an agent that inhibits cap-dependent translation is an agent that inhibits eIF4E. In some embodiments, an agent that inhibits eIF4E is an eIF4E inhibitor. In some embodiments, the eIF4E inhibitor is selected from 4E1RCat, 4E2RCat, LY2275796, SBI-756 and 4EGI-1. In some embodiments, the eIF4E inhibitor is selected from 4E1RCat, 4E2RCat, LY2275796, SBI-756 and 4EGI-1. In some embodiments, the eIF4E inhibitor is 4EGI-1.

In some embodiments, an agent that inhibits cap-dependent translation is an agent that inhibits MNK. In some embodiments, an agent that inhibits MNK is an MNK inhibitor. In some embodiments, the MNK inhibitor is selected from CGP57380, BAY1143269, ETC-206, MNK-11, MNK-12, MNK1/2-IN-7, SEL201, Cercosporamide, MNKI-8e and Tomivosertib. In some embodiments, the MNK inhibitor is selected from MNKI-8e and Tomivosertib. In some embodiments, the MNK inhibitor is Tomivosertib.

In some embodiments, an agent that modulates translation is an agent that accelerates translation elongation. In some embodiments, an agent that accelerates translation elongation is an agent that inhibits eEF2K. In some embodiments, an agent that inhibits eEF2K is an eEF2K inhibitor. In some embodiments, the eEF2K inhibitor is selected from NH125, rottlerin, and A-484954. In some embodiments, the eEF2K inhibitor is A-484954.

In some embodiments, an agent that modulates translation is an agent that inhibits translation termination. In some embodiments, an agent that inhibits translation termination is an agent that inhibits eRF1. In some embodiments, an agent that inhibits translation termination is an agent that degrades eRF1. In some embodiments, an agent that inhibits translation termination is an agent that depletes eRF1. In some embodiments, an agent that inhibits translation termination is an eRF1 inhibitor. In some embodiments, the eRF1 inhibitor is selected from SRI-41315, Apidaecin, dimethyloxalylglycine (DMOG) and N-oxalylglycine (NOG). In some embodiments, the eRF1 inhibitor is selected from SRI-41315, Apidaecin and NOG. In some embodiments, the eRF1 inhibitor is selected from Apidaecin and NOG. In some embodiments, the eRF1 inhibitor is SRI-41315. In some embodiments, the eRF1 inhibitor is Apidaecin. In some embodiments, the eRF1 inhibitor is NOG.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

Cell culture: WT and TSC−/− MEFs, APC 1450X cells, and human colon carcinoma cell lines were cultured in Dulbecco's modified Eagle's medium (DMEM) supplemented with 10% fetal calf serum (FCS) and 100 U/ml penicillin-streptomycin. DU4475 were cultured in Roswell Park Memorial Institute (RPMI) 1640 Medium with 10% FCS and 100 U/ml penicillin-streptomycin. Cells were kept in a humidified 5% CO2 atmosphere at 37° C. All the following cell lines were from ATCC: COLO 320-ATCC CCL-220, SW403-ATCC CCL-230, SW620-ATCC CCL-227, SW837-ATCC CCL-235, DU4475-ATCC HTB-123, HCT116-ATCC CCL-247, and SW1417-ATCC CCL-238.

Antibodies and reagents: The following antibodies and reagents were used: anti-GFP (mouse monoclonal; Santa Cruz; sc-9996, 1:750), anti-APC (rabbit polyclonal; Santa Cruz; sc-7930, 1:500), anti-active β-catenin (rabbit polyclonal; Cell Signaling Technology; D2U8Y, 1:2000), anti-tubulin (mouse monoclonal; Sigma; T6199, 1:10,000), Anti-p-S6K1 (rabbit polyclonal; Cell Signaling Technology; #9205, 1:1000), Anti-S6K1 (rabbit polyclonal; Cell Signaling Technology; #9202, 1:1000), Anti-4EBP-1 (rabbit polyclonal; Abcam, ab2606, 1:1000), Anti-p-S6 rabbit polyclonal; Cell Signaling Technology; #2211, 1:1000), Anti-p-eIF4E (rabbit polyclonal; Cell Signaling Technology; #9741, 1:1000), Anti-TSC2 (rabbit polyclonal; Cell Signaling Technology; #4308, 1:1000), anti-mouse and anti-rabbit-HRP (Jackson Laboratories, 1:10,000), Gentamicin sulfate (Biological Industries,03-035), G418 sulfate (Mercury-ltd, CAS 108321-42-2), Rapamycin (AdooQ BioScience, A10782) in DMSO, Torin-1 (Caymanchem, 10997) in DMSO, PF-4708671 (AdooQ BioScience, A11755) in DDW, and 4EGI-1 (AdooQ BioScience, A14199) in DMSO, Tomivosertib (caymanchem, 21957) in DMSO, A-484954 (caymanchem, 28279) in DMSO, SRI-41315 was dissolve in DMSO, Apidaecin IB (Anaspec, AS-62044) in DDW, N-Oxalylglycine (caymanchem, 13944) in DMSO, Escin (Santa Cruz, SC-221596) in methanol, Ataluren (Caymanchem, 16758) in DMSO and Erythromycin (Caymanchem, 16486) in ethanol.

Immunofluorescence: Cells were grown on 13 mm round coverslips and then fixed for 20 min in PBS containing 4% paraformaldehyde. After three washes with PBS, the fixed cells were permeabilized with 0.1% Triton X-100 for 10 min and blocked with bovine serum albumin for 1 h. Subsequently, cells were incubated at room temperature with rabbit anti-active β-catenin (1:250) and Alexa fluor 488 anti-Rabbit (1:500) for 60 and 45 min, respectively. 4′,6-Diamidino-2-phenylindole (DAPI, Sigma 10 μg/ml) was used to stain cell nuclei. An independent script was used to quantify the RGB intensity of nuclear β-catenin.

Western blot analysis: Cells were washed with PBS and solubilized in lysis buffer (50 mM Tris pH 7.5, 100 mM NaCl, 1% Triton X-100, 2 mM EDTA) containing a protease inhibitor cocktail (Sigma). Full-length APC was detected by solubilizing CRC cell lines in 6 M urea lysis buffer (50 mM Tris pH 7.5, 120 mM NaCl, 1% NP-40, 1 mM EDTA) containing protease inhibitor cocktail. Extracts were clarified by centrifugation at 12,000×g for 15 min at 4° C. Following SDS polyacrylamide gel electrophoresis (SDS-PAGE), proteins were transferred to nitrocellulose membranes and blocked with 5% low-fat milk. The membranes were then incubated with specific primary antibodies, washed with PBS containing 0.001% Tween-20 (PBST), and incubated with the appropriate horseradish peroxidase-conjugated secondary antibody. After washing in PBST, membranes were subjected to enhanced chemiluminescence detection analysis. Band intensities were quantified by Fusion-Capt analysis software.

RNA isolation and RT-qPCR analysis: Total RNA was isolated from the cultured cells using TRI reagent (Bio-lab) and an RNA extraction kit (ZYMO) according to the manufacturer's protocol. Total RNA (1 ug) was reverse transcribed using the iScript cDNA Synthesis Kit (Bio-Rad) according to the manufacturer's instructions. Real-time PCR was performed using the CFX Connect Real-Time PCR Detection System (Bio-Rad) using a SYBR Green Master mix (PCR Biosystems). Actin was used as a housekeeping control. All reactions were in triplicates. The primers for the amplification of the specific cDNA sequences are provided in Table 1.

TABLE 1
qPCR primers:

| | Fw primer (SEQ ID NO:) | Rv primer (SEQ ID NO:) |
|---|---|---|
| APC | 5′ GCTCTATGAAAGGCTGCATGAG 3′ | 5′ TCACACTTCCAACTTCTCGC 3′ |
| | (1) | (2) |
| ACTIN | 5′ CCTGGCACCCAGCACAAT 3′ | 5′ GGGCCGGACTCGTCATACT 3′ |
| | (3) | (4) |

Cell viability assay: PrestoBlue viability reagent (Thermofisher, A13261) was used according to the manufacturer's protocol. Measurement of absorbance at 570 and 600 nm, using Epoch microplate Spectrophotometer (BioTek). All the treatments were in triplicates.

Statistical analysis: Data were analyzed using GraphPad Prism software and are presented as the mean with standard deviation. Analysis of variance (ANOVA) was performed when appropriate to assess the significance of variations using Tukey's multiple comparisons. P values are as indicated.

### Example 1: The mTOR Substrate 4EBP-1 Mediates the Antibiotic-Mediated Readthrough Response to Serum Starvation in Cancerous Cell Lines

It has previously been shown that stress induced by serum starvation increases antibiotic-mediated nonsense mutation readthrough using both a reporter-based cellular system and the endogenous APC gene product in the CRC cell line Colo320 (see Wittenstein, et al., “Serum starvation enhances nonsense mutation readthrough”, J Mol Med (Berl), (2019), the contents of which are hereby incorporated by reference in their entirety). To understand the physiological mechanisms underlining this effect, whether serum starvation may enhance antibiotic-mediated readthrough in additional cell lines (harboring different endogenous APC nonsense mutations) was tested. Cells were incubated for 24 h in a medium containing 10% or 1% serum supplemented with 1.5 mg/ml G418. The results show that antibiotic-mediated nonsense mutation readthrough was increased in three (SW837, SW620, and DU4475) out of the four tested cell lines when the serum concentration was reduced (FIG. 1A). The APC tumor suppressor is an essential component of a cytoplasmic protein complex that targets β-catenin for destruction. Thus, the functionality, at least partly, of the restored APC was demonstrated by its ability to reduce the levels of active β-catenin (FIG. 1A; middle blot). Unlike the results obtained using the Colo320 cells, despite APC enhanced restoration, active β-catenin levels were not further reduced following serum depletion. Different studies have shown that the levels of β-catenin do not directly correlate to the levels of full-length APC. This phenomenon is thought to represent a threshold of β-catenin expression. Importantly, it was shown that the number of β-catenin binding sites in the APC protein affects its ability to decrease β-catenin levels, though not in an absolute correlation. Our results using the Colo320 cells in which, uniquely, the APC has no β-catenin binding sites show the strongest effect on-catenin expression levels. For example, SW837 and SW620 still retain β-catenin binding sites, and thus restored full-length APC may not directly affect the expression of β-catenin. Thus, in most experiments (FIG. 2-5), the Colo320 cells were used. In addition, other mechanisms, such as neddylation, GSK3ß mTOR-dependent phosphorylation, and microRNAs' expression, that have been shown to be involved in regulating β-catenin levels may be influenced by the readthrough-inducing drugs and restrict the effects on β-catenin.

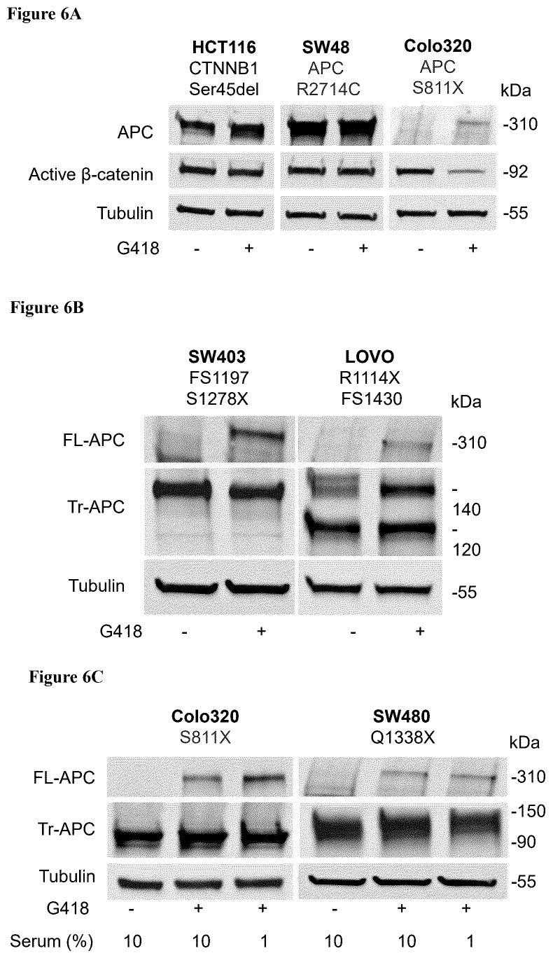

The antibiotic-mediated nonsense mutation readthrough specificity was confirmed by comparing CRC cell lines without an APC nonsense mutation to the Colo320 cells (FIG. 6A). Indeed, HCT116, which encodes for a full-length APC protein, and SW48, which carries an APC missense mutation, were not affected by the G418 treatment, and active β-catenin protein levels were unchanged (FIG. 6A). Moreover, neither G418 nor serum deprivation had any effect on the truncated APC (Tr-APC) protein expressed in the different cell lines tested (FIG. 6B-6C).

To understand why reduced serum levels enhanced antibiotic-mediated readthrough only in certain cell lines, a two-class comparison of protein expression levels using the Broad Institute DepMap web portal was performed using the cell lines that responded to serum depletion (Colo320, SW837, SW620, and DU4475) (FIG. 1A) compared to the non-responsive cells (SW1417, SW403, LoVo, SW480) (FIG. 1A). The analysis results indicated statistically significant differences for 8 out of a total of 214 proteins, with the greatest differences noted for 4E (eIF4E)-binding protein 1 (4EBP-1) and its phosphorylated forms (FIG. 1B). 4EBP-1 belongs to a family of translation repressor proteins and is a known substrate of the mammalian target of the rapamycin (mTOR) signaling pathway. The mTOR pathway controls, among other things, the cell response to nutrients. Activated mTOR complex1 (mTORC1) signaling leads to the phosphorylation of several downstream components, including S6K1 and 4EBP-1. When stimulated, mTORC1 mediates the phosphorylation of 4EBP-1 to initiate protein synthesis. In our analysis, 4EBP-1 and its three phosphorylated forms were highly expressed in the cells that demonstrated increased antibiotic induced-readthrough of nonsense mutations following serum depletion (FIG. 1C).

### Example 2: MTOR Inhibition Increases Antibiotic-Induced Nonsense Mutation Readthrough

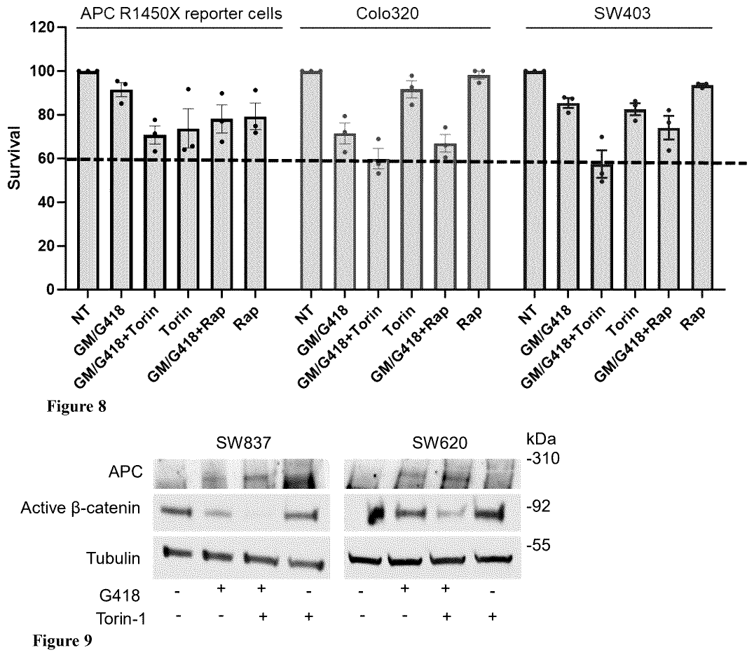

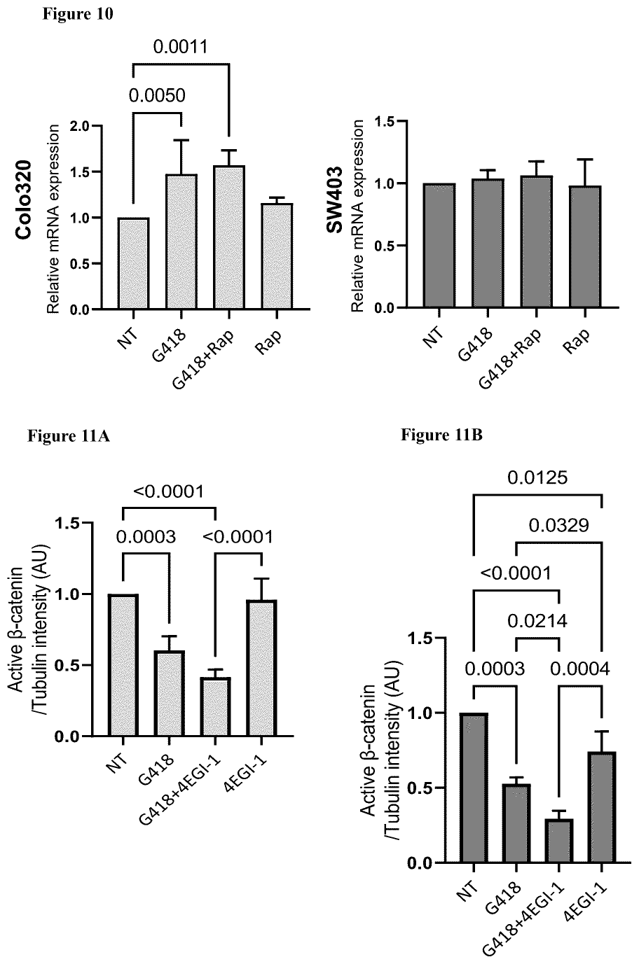

mTOR is a highly conserved serine/threonine kinase complex that controls cell growth and metabolism. The protein complex controls the levels of available cellular energy substrates and maintains the available amino acid pool by regulating protein translation. Dysregulation of the mTOR pathway leads to aberrant protein translation and various pathological conditions. When amino acids are scarce (as in the case of serum starvation), mTOR turns off, and protein production is reduced. The involvement of the mTOR cascade in antibiotic-mediated nonsense mutation readthrough was assessed by examining the effect of the tuberous sclerosis complex 1/2 genes (TSC1/2) that negatively regulates mTOR signaling. Immortalized TSC1/2−/− mouse embryo fibroblasts (MEFs), which have a constitutively high mTOR activity, were transfected with a nonsense sequence readthrough-sensitive reporter plasmid GFP-BFP that harbors specific stop codon (and surrounding sequences) inserted between the GFP and BFP open reading frames. The upstream GFP protein serves as a control for total chimeric protein expression, while the levels of GFP-BFP fusion protein products produced reflect the degree of stop codon readthrough activity and can be measured by western blot analysis. The effect of Gentamycin (GM) on readthrough in TSC1/2−/− was compared to that in wild-type (WT) MEFs. Interestingly, antibiotic treatment of the TSC1/2−/− cells did not induce readthrough (FIG. 2A), and thus, no fusion GFP-BFP fusion protein was detected, although the wild-type cells did express the GFP-BFP fusion protein following treatment. These results suggest that the mTOR pathway may be involved in antibiotic-mediated nonsense mutation readthrough. This notion is supported by the observation that treating TSC1/2−/− cells with the mTOR inhibitor Rapamycin (Rap) increases the ability of the antibiotic to induce stop codon readthrough (FIG. 2A). To further explore the involvement of the mTOR pathway in antibiotic-mediated nonsense mutation readthrough; two known inhibitors of the mTOR cascade: Torin-1 (FIG. 7) and Rapamycin (FIG. 2) were tested. Torin-1 is a synthetic mTOR inhibitor that blocks ATP-binding to mTOR and thus inactivates both mTORC1 and mTORC2, whereas Rapamycin is a macrolide known to inhibit cap-dependent mRNA translation, selectively targeting mTORC1. To decrease toxicity-related stress, the antibiotic concentration was reduced to 500 μg/ml (cell survival of 60-100%; FIG. 8). Treating cells stably expressing the GFP-BFP reporter plasmid (APC R1450X) with either Torin-1 or Rapamycin, in the presence of the GM, enhanced antibiotic-mediated PTC readthrough (FIGS. 7A and 7B, respectively). The effect of mTOR inhibition was next examined in the CRC cell line Colo320, where serum starvation was shown to enhance G418-mediated readthrough. Treated Colo320 cells demonstrated relatively high levels of APC restoration following antibiotic treatment, and importantly, APC readthrough was further increased in response to mTOR inhibition (Torin-1; FIG. 7B or rapamycin; FIG. 2C). The levels of active β-catenin were reduced accordingly indicating the functionality of the restored APC protein. Resembling the serum starvation effect, SW403 cells did not respond to mTOR inhibition, and the APC restoration levels were similar to those induced by the antibiotic alone (Torin-1; FIG. 7C or rapamycin; FIG. 2D). This correlation between the response to mTOR inhibition and serum starvation was also seen in other cell lines (FIG. 9). FIG. 2E demonstrates that, as expected, rapamycin addition impeded the nuclear translocation of β-catenin only in Colo320 cells. Interestingly, although mutated APC transcripts are relatively stable, as they often escape NMD, a slight increase in mRNA levels was observed in treated Colo320 cells compared to mRNA transcripts in the SW403 cells that were unaffected by readthrough or readthrough enhancement (FIG. 10).

### Example 3: Reduced Cap-Dependent Translation Initiation Increases Antibiotic-Mediated Nonsense Mutation Readthrough

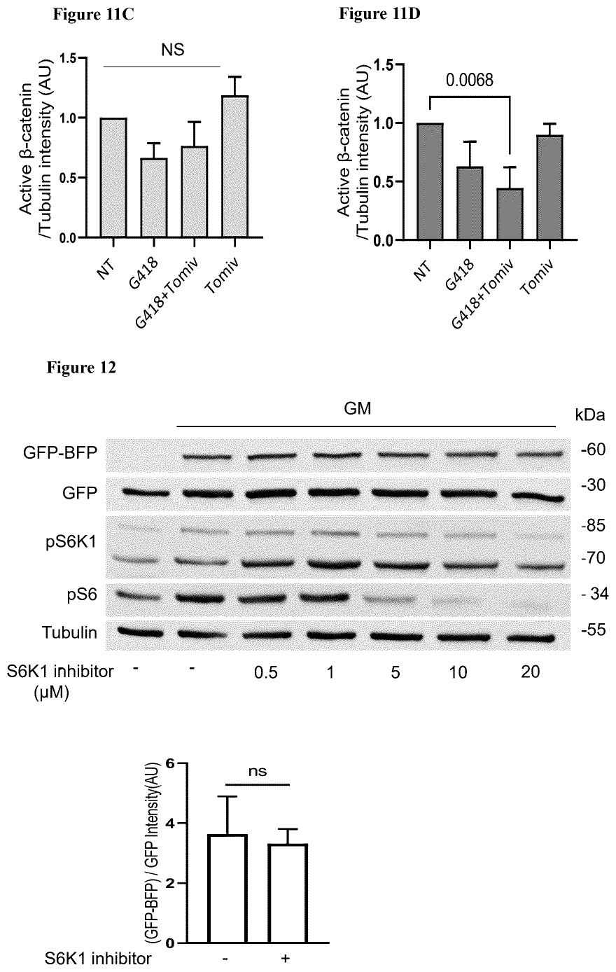

4EBP-1 is a known substrate of the mTOR pathway, and its un-phosphorylated form actively inhibits protein translation initiation. The translation initiation factors 4G (eIF4G), 4E (eIF4E), and 4A (eIF4A) form a protein complex, which regulates the instigation of protein translation. Un-phosphorylated, 4EBP-1 binds to eIF4E, preventing its association with eIF4G, thus inhibiting protein translation. The small-molecule inhibitor 4EGI-1, stabilizes the eIF4E/4EBP-1 bond, which hinders the eIF4E-eIF4G interaction, leading to translation initiation inhibition. Our data demonstrate that 4EGI-1 enhances antibiotic-induced nonsense mutation readthrough of both the GFP-BFP fusion protein in the APC R1450X reporter cell line (FIG. 3A) and the endogenous APC protein in Colo320 CRC cells (FIG. 3B). Interestingly, similar results were observed in the SW403 CRC cell line (FIG. 3C), although this cell line falls into the cell group in which serum starvation did not affect readthrough levels. This result may indicate that directly inhibiting cap-dependent translation initiation regardless of serum levels is sufficient for enhancing antibiotics-induced nonsense mutation readthrough. The Inventors thus tested an additional inhibitor that targets translation initiation. Translational control is mediated by a 7-methyl-GTP cap structure present at 5′ termini of eukaryotic mRNAs. eIF4E plays a crucial role in cap-dependent mRNA translation initiation as it binds to the cap structure and positions the ribosome near the 5′ terminus of the mRNA. MAPK-interacting kinase (MNK) phosphorylates eIF4E at Ser209, using eIF4G as a docking site. The phosphorylation of eIF4E increases its affinity for the cap of mRNA and potentially facilitates its entry into the initiation complexes. Tomivosertib, a potent and highly selective dual MNK 1/2 inhibitor, was used to impede eIF4E phosphorylation and inhibit translation initiation. Similarly to 4EGI-1, MNK inhibition enhances antibiotic-induced PTC readthrough of the GFP-BFP fusion protein in the APC R1450X reporter cell line (FIG. 3D). The expression levels of full-length endogenous APC in both the Colo320 (FIG. 3E) and SW403 CRC cell lines (FIG. 3F) were increased in response to Tomivosertib (when combined with G418). The effects of APC restoration on active β-catenin levels are shown in FIG. 11A-11D (4EGI-1; FIG. 11A-11B, Tomivosertib; FIG. 11C-11D).

S6K1 phosphorylation is also a known stimulus of protein synthesis; however, our results indicate that inhibiting S6K1 (using the S6K1 inhibitor (PF-4708671) did not affect antibiotic-mediated nonsense suppression in the APC R1450X reporter cell line (FIG. 12). Interestingly, since Colo320 and SW403 do not express p-S6K1 (FIG. 2C-D, 7B-C), it was concluded that S6K1 is not involved in the effects of mTOR inhibition on antibiotic-mediated nonsense mutation readthrough.

### Example 4: Targeting Different Stages of the Protein Translation Process Enhances Antibiotic-Mediated Nonsense Codon Readthrough

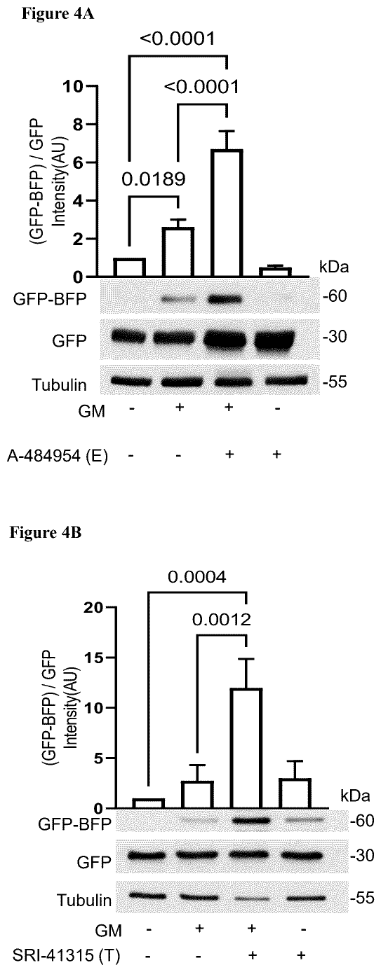

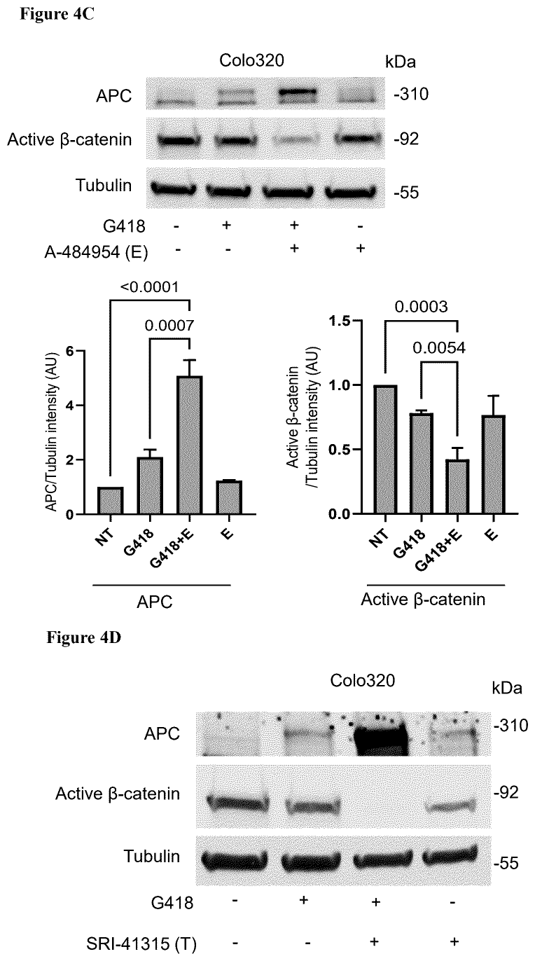

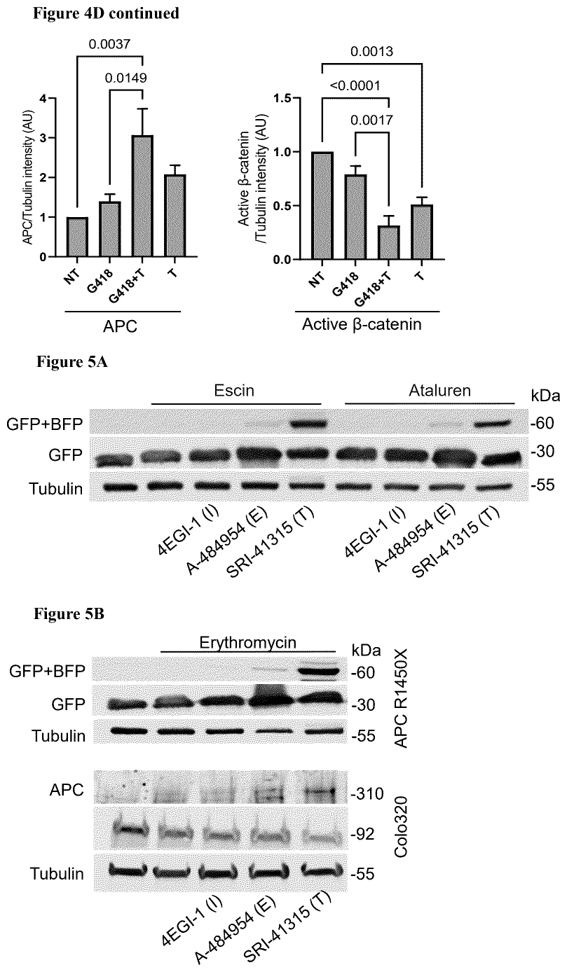

During mRNA translation, aminoacyl-tRNAs are recruited into the aminoacyl (A) site by eukaryotic elongation factor 1A (eEF1A). Next, the eukaryotic elongation factor 2 (eEF2) GTPase, which is regulated by the highly specific eEF2 protein kinase (eEF2K), induces translation elongation. Inactivating eEF2K enhances eEF2 activity leading to accelerated elongation that is usually accompanied by impaired translational fidelity. To test the effect of elongation rate on readthrough activity, A-484954, a highly selective eEF2K inhibitor, was used. As shown in FIGS. 4A and 4C, eEF2K inhibition enhances antibiotic-induced nonsense mutation readthrough of both the GFP-BFP fusion protein in the APC R1450X reporter cell line (FIG. 4A) and endogenous APC in Colo320 (FIG. 4C). This result indicates that accelerated elongation can increase antibiotic mediated nonsense mutation readthrough.

Next, the Inventors examined whether targeting translation termination may also influence antibiotic-induced PTC. Translational readthrough efficiency depends on competition between stop codon recognition by eukaryotic translation termination factor 1 (eRF1) and decoding of the stop codon by a near-cognate tRNA. SRI-41315 induces translational readthrough by depleting eRF1 levels through a proteasome-mediated degradation pathway. It was demonstrated that SRI-41315 enhances antibiotic-induced nonsense mutation readthrough of both the GFP-BFP fusion protein in the APC R1450X reporter cell line (FIG. 4B) and endogenous APC in Colo320 (FIG. 4D). SRI-41315 treatment alone induced low levels of nonsense mutation readthrough in both experiments (FIG. 4B, 4D).

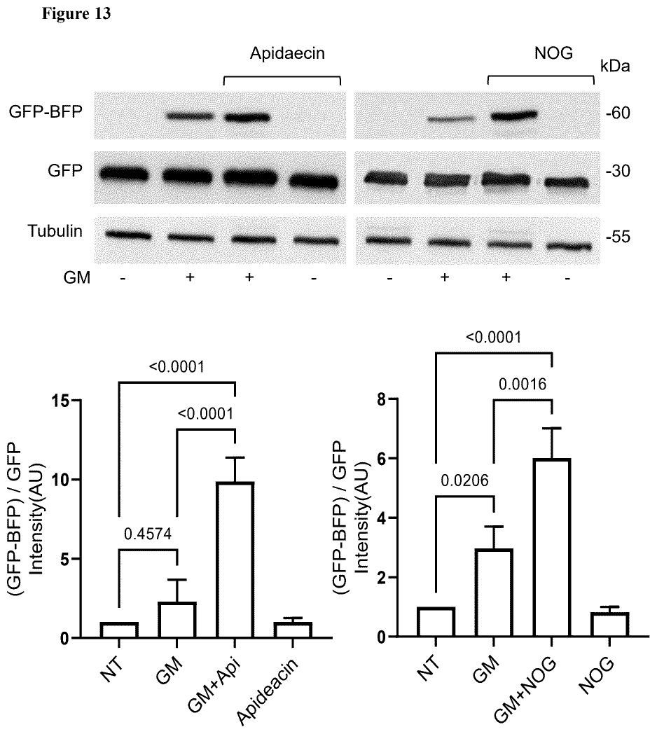

Apidaecin, an 18-amino acid proline-rich antimicrobial peptide from honeybees, was recently shown to have RF1- and RF2-inhibiting activity in bacteria. An additional eRF1 inhibitor is NOG (dimethyloxalylglycine; DMOG), which reduces the hydroxylation of newly synthesized eRF1. Inhibition of eRF1 regular activity by Apidaecin or NOG-induced antibiotic-induced nonsense mutation readthrough of the GFP-BFP chimeric protein in APC R1450X reporter cell line (FIG. 13).

### Example 5: Targeting Different Stages of the Protein Translation Process Enhances Readthrough Mediated by Non-Aminoglycosides

Taken together, it was concluded that the modulation of different stages of the protein translational machinery affects the general mechanism of antibiotic-mediated nonsense suppression. To determine whether this is specific to aminoglycosides, additional non-aminoglycoside inducers were tested in both the reporter system and in Colo320 CRC cells. The Inventors used Erythromycin, a macrolide that was shown to induce nonsense suppression in several systems, including a clinical trial, Escin, a natural herbal product that was shown to induce readthrough, and Ataluren (PTC124), which is a benzoic acid derivative that was shown to induce readthrough in various syndromes and model systems. Ataluren was recently approved to treat DMD patients carrying nonsense mutations. As shown in FIG. 5, the effects of all readthrough-enhancing agents were enhanced by modifying the protein translation process. These results confirm that enhanced nonsense suppression by manipulating different translation steps is a general mechanism that can be applied to induce readthrough mediated by both aminoglycosides and non-aminoglycosides compounds.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of increasing readthrough of a premature termination codon (PTC) by a readthrough-inducing agent in a cell, the method comprising modulating translation in said cell, wherein said modulating translation comprises at least one of: inhibiting cap-dependent translation initiation in said cell, accelerating translation elongation in said cell, and inhibiting translation termination in said cell, thereby increasing readthrough of a PTC by a readthrough-inducing agent.

2. The method of claim 1, wherein said readthrough-inducing agent is an antibiotic.

3. The method of claim 2, wherein said antibiotic is an aminoglycoside selected from the group consisting of: geneticin, gentamicin, paromomycin and tobramycin.

4. The method of claim 2, wherein said antibiotic is selected from the group consisting of: erythromycin, azithromycin, spiramycin, josamycin, and tylosin.

5. The method of claim 1, wherein said readthrough-inducing agent is selected from escin, ELX-02, NPC-14, 2,6-diaminopurine (DAP), CC-90009, amlexanox and ataluren.

6. The method of claim 1, further comprising contacting said cell with said readthrough-inducing agent, wherein said readthrough inducing agent is selected from the group consisting of: geneticin, gentamicin, paromomycin, tobramycin, erythromycin, azithromycin, spiramycin, josamycin, tylosin, escin, ELX-02, NPC-14, 2,6-diaminopurine (DAP), CC-90009, amlexanox and ataluren.

7. The method of claim 1, wherein said modulating translation comprises inhibiting cap-dependent translation initiation and comprises contacting said cell with a mammalian target of rapamycin (mTOR) inhibitor, a Eukaryotic translation initiation factor 4E (eIF4E) inhibitor, a Mitogen-activated protein kinase interacting kinase (MNK) inhibitor, or a combination thereof.

8. The method of claim 7, wherein said inhibiting cap-dependent translation initiation does not comprise administering a Ribosomal protein S6 kinase beta-1 (S6K1) inhibitor;

9. The method of claim 1, wherein said modulating translation comprises accelerating translation elongation and comprises contacting said cell with a eukaryotic elongation factor 2 kinase (eEF2K) inhibitor.

10. The method of claim 1, wherein said inhibiting translation termination comprises contacting said cell with a eukaryotic translation termination factor 1 (eRF1) inhibitor.

11. The method of claim 7, wherein at least one of:
a. said mTOR inhibitor is selected from the group consisting of: everolimus, temsirolimus, ridaforolimus, umirolimus, zotarolimus, AZD8055, PP242, INK128, WYE-354, OSI-027 and KU-0063794, rapamycin and Torin-1;
b. said eIF4E inhibitor is selected from the group consisting of: 4E1RCat, 4E2RCat, LY2275796, SBI-756 and 4EGI-1; and
c. said MNK inhibitor is selected from the group consisting of: CGP57380, BAY1143269, ETC-206, MNK-11, MNK-12, MNK1/2-IN-7, SEL201, Cercosporamide, MNKI-8e and Tomivosertib.

12. The method of claim 9, wherein said eEF2K inhibitor is selected from NH125, rottlerin, and A-484954.

13. The method of claim 10, wherein said eRF1 inhibitor is selected from SRI-41315, Apidaecin, dimethyloxalylglycine (DMOG) and N-oxalylglycine (NOG).

14. A method of treating a disease characterized by a pathogenic premature termination codon (PTC) in a disease-associated gene in a subject in need thereof, the method comprising increasing readthrough of said PTC by a readthrough-inducing agent in a cell of said subject by a method of any one of claim 1, thereby treating a disease characterized by a pathogenic PTC.

15. The method of claim 14, further comprising selecting a subject suffering from a disease characterized by a pathogenic PTC in a disease-associated gene.

16. The method of claim 14, wherein
a. said disease is cancer and said disease-associated gene is a tumor suppressor gene selected from the group consisting of: adenomatous polyposis coli (APC), Ataxia-telangiectasia mutated (ATM), Breast cancer type 1 susceptibility protein (BRCA1), BRCA2, cadherin-11 (CDH1), cyclin-dependent kinase inhibitor 2A (CDKN2A), Menin (MEN1), Neurofibromin (NF1), merlin (NF2), Protein patched homolog 1 (PTCH1), Phosphatase and tensin homolog (PTEN), retinoblastoma protein (RB1), SMAD family member 4, Mothers against decapentaplegic homolog 4 (SMAD4), Serine/threonine kinase 11 (STK11), p53 (TP53), tuberous sclerosis 1 (TSC1), TSC2, Von Hippel-Lindau tumor suppressor (VHL), and Wilms tumor protein (WT1);
b. said disease is cystic fibrosis and said disease-associated gene is cystic fibrosis transmembrane conductance regulator (CFTR);
c. said disease is muscular dystrophy and said disease-associate gene is dystrophin (DMD);
d. said disease is nephropathic cystinosis and said disease-associated gene is cystinosin (CTNS);
e. said disease is epidermolysis bullosa and said disease-associated gene is laminin beta 3 (LAMB3);
f. said disease is hereditary hypotrichosis simplex and said disease-associated gene is Lysophosphatidic acid receptor 6 (LPAR6/P2RY5) or lipase H (LIPH); or
g. said disease is adenomatous polyposis and said disease-associated gene is APC.

17. The method of claim 16, wherein said muscular dystrophy is Duchenne Muscular Dystrophy (DMD).

18. The method of claim 14, further comprising administering to said subject said readthrough-inducing agent, wherein said readthrough inducing agent is selected from the group consisting of: geneticin, gentamicin, paromomycin, tobramycin, erythromycin, azithromycin, spiramycin, josamycin, tylosin, escin, ELX-02, NPC-14, 2,6-diaminopurine (DAP), CC-90009, amlexanox and ataluren.

19. A kit comprising:
a. a readthrough-inducing agent, wherein said readthrough inducing agent is selected from the group consisting of: geneticin, gentamicin, paromomycin, tobramycin, erythromycin, azithromycin, spiramycin, josamycin, tylosin, escin, ELX-02, NPC-14, 2,6-diaminopurine (DAP), CC-90009, amlexanox and ataluren.; and
b. an agent that modulates translation, wherein said agent that modulates translation is a mammalian target of rapamycin (mTOR) inhibitor, a Eukaryotic translation initiation factor 4E (eIF4E) inhibitor, a Mitogen-activated protein kinase interacting kinase (MNK) inhibitor, a eukaryotic elongation factor 2 kinase (eEF2K) inhibitor, a eukaryotic translation termination factor 1 (ERF1) inhibitor, or a combination thereof.

20. The kit of claim 19, wherein at least one of:
a. said mTOR inhibitor is selected from the group consisting of: everolimus, temsirolimus, ridaforolimus, umirolimus, zotarolimus, AZD8055, PP242, INK128, WYE-354, OSI-027 and KU-0063794, rapamycin and Torin-1;
b. said eIF4E inhibitor is selected from the group consisting of: 4E1RCat, 4E2RCat, LY2275796, SBI-756 and 4EGI-1;
c. said MNK inhibitor is selected from the group consisting of: CGP57380, BAY1143269, ETC-206, MNK-I1, MNK-12, MNK1/2-IN-7, SEL201, Cercosporamide, MNKI-8e and Tomivosertib;
d. said eEF2K inhibitor is selected from NH125, rottlerin, and A-484954; and
e. said eRF1 inhibitor is selected from SRI-41315, Apidaecin, dimethyloxalylglycine (DMOG) and N-oxalylglycine (NOG).


SEQUENCE LISTING (4 sequences)

SEQ ID NO: 1 - DNA, 22 bp, synthetic construct
  source 1..22: mol_type = other DNA
gctctatgaaaggctgcatgag

SEQ ID NO: 2 - DNA, 20 bp, synthetic construct
  source 1..20: mol_type = other DNA
tcacacttccaacttctcgc

SEQ ID NO: 3 - DNA, 18 bp, synthetic construct
  source 1..18: mol_type = other DNA
cctggcacccagcacaat

SEQ ID NO: 4 - DNA, 19 bp, synthetic construct
  source 1..19: mol_type = other DNA
gggccggactcgtcatact